(19) United States
(12) Patent Application Publication
(10) Pub. No.: US 2004/0109840 A1
(43) Pub. Date: Jun. 10, 2004

(54) Non-rinse composition for combing hair, that imparts lightness to hair

(76) Inventors: Jose Carlos de Azevedo, Sao Jose dos Campos (BR); Silvana Masiero, Sao Jose dos Campos (BR)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/671,211

(22) Filed: Sep. 25, 2003

(30) Foreign Application Priority Data
Sep. 25, 2002 (BR) PI0203885-4

(51) Int. Cl. A61K 7/06; A61K 7/11; A61K 7/075; A61K 7/08

(52) U.S. Cl. 424/70.27; 424/70.31; 424/70.12

(57) ABSTRACT

The present invention discloses a no-rinse composition for combing hair, which imparts lightness to hair, leaving the, after brushing, with an appearance of loose hair, with well-shaped curls, clean, with an adequate volume and untangled, thus facilitating the combing.

The basic formulation of the no-rinse composition of the present invention comprises:

(i) a thickener;

(ii) a fatty alcohol+surfactant mixture at low concentration;

and it may further comprise:

(iii) a cationic polymer; and

(iv) a soluble or insoluble silicone.

The present invention also provides a non-rinse composition, with the following formulation:

(i) a thickener; and

(iii) a cationic polymer.

This application claims the benefit of priority of Brazilian Patent Application No. PI 0203885-4, which is incorporated herein by reference.

### FIELD OF THE INVENTION

The present invention relates to a composition for combing hair without rinsing, which imparts lightness to the hair, leaving them, after brushing, with the appearance of looseness, with well-shaped, clean, untangled curls having adequate volume, even in wetting days wherein the tendency of the hair is to “built up”.

### BACKGROUND OF THE INVENTION

The prior art discloses creams for combing hair without rinsing, with a formulation based on a mixture of fatty alcohol+cationic surfactant. The result of this mixture is a “heavy” formula due to the presence, at high concentration, of both fatty alcohol and surfactant, in order to obtain a viscosity suitable for such composition. The disadvantages of the “heavy” formula are: bad appearance of the hair, such as dirt, oiliness, which makes the hair dwindled and heavy, besides not allowing one to make curls.

Another disadvantage disclosed in prior art formulations of this type is the fact that the fatty alcohol and the cationic surfactant are used separately in the process for preparing the non-rinse formulation.

Since the fatty alcohol is usually commercialized in the form of flakes or scales, it is necessary to be heated and melted in order to be further added to the process together with the cationic surfactant. This procedure drives to a high power consumption by the manufacturer.

Non-rinse formulations obtained from the use of common thickeners to reach the adequate viscosity are also known. However, such mixture drives hair to either present undesirable characteristics as already previously mentioned, in addition to the fact that, after hair drying, a film is formed which enfolds hair's strand, and that subsequently itself leaves causing the uncomfortable appearance of dandruff and/or dirtiness.

Patent application PI 9605799-8, corresponding to U.S. Pat. No. 6,221,347 B1 and to patent application WO 96/23482, discloses non-washing cosmetic compositions containing, in a cosmetically acceptable medium, at least one nonionic guar gum and at least one non-crosslinked anionic polymer. This document also discloses a process for the cosmetic treatment of keratin substances, such as hair or eyelashes, characterized in that it consists in applying a cosmetic composition as defined above to the keratin substances, and then possibly in rinsing with water.

Patent application U.S. Ser. No. 2001/014,344, published on Aug. 16, 2001, discloses non-washing cosmetic compositions containing, in a cosmetically acceptable medium, at least one nonionic guar gum and at least one non-crosslinked anionic polymer. This application further discloses a process for the cosmetic treatment of keratin substances, such as the hair or the eyelashes, characterized in that it consists in applying a cosmetic composition as defined above to the keratin substances, and then possibly in rinsing with water.

### OBJECTIVES OF THE INVENTION

One of the objectives of the invention is to provide a non-rinse composition containing effective amounts of a thickener, of the chemical class of synthetic polymers, and a mixture of fatty alcohol+cationic surfactant at low concentration. The function of said thickener is to act as an aqueous agent for intensifying viscosity, which allows the reduction of the amount of the fatty alcohol+cationic surfactant mixture used.

It is a further objective of the present invention the use of a combination of fatty alcohol+cationic surfactant, already previously mixed, that provides said process to be carried out completely in a cold way, reducing, therefore, the costs of the process and the power consumption.

Is yet another objective of the present invention to provide a non-rinsing composition containing effective amounts of a thickener, of the chemical class of the synthetic polymers, of a mixture of fatty alcohol+cationic surfactant at low concentration, and of a cationic polymer.

Is yet another objective of the present invention is to use effective amounts of a thickener, of a cationic polymer, of a mixture of fatty alcohol+cationic surfactant and of a silicone for preparing a non-rinse composition.

It is also an objective the present invention to provide a method of imparting softness to the hair by applying the non-rinse composition disclosed by the present invention.

### SUMMARY OF THE INVENTION

The present invention relates to “leave-on” or “leave-in” type non-rinse compositions with a low % of fatty alcohol and soluble cationic surfactant, containing a special thickener for imparting ideal viscosity to the product. In particular, the present invention is related to non-rinse combing creams, which may be used when the hair is dry and not only humid, and which do not imparts a heavy and falling appearance.

According to one of the variants of the present invention, composition comprising a thickener in combination with a cationic polymer.

The composition of the present invention may by applied to any type of hair, preferably to curled hair.

The basic formulation of the no-rinse composition of the present invention comprises:

(i) a thickener of the group of hydrophobic non-ionic modified polymers;

(ii) a fatty alcohol+surfactant mixture at low concentration;

and it may further comprise:

(iii) a cationic polymer; and

(iv) a soluble or insoluble silicone.

The present invention also provides a non-rinse composition, with the following formulation.

(i) a thickener of the group of hydrophobic non-ionic modified polymers; and

(ii) a cationic polymer.

In order to achieve the thickener suitable for the formulation of the no-rinse composition of the present invention, the inventors aimed at providing a formulation having as its main characteristic a product that, after being applied onto the hair, would not provide an appearance of heavy hair, thus preventing curl formation. Another aspect is that the thickener would not cause an appearance of “falling” hair, which would not cause the film on the hair to loosen (thus causing an uncomfortable appearance of dandruff).

It was then found that certain groups of thickeners were capable of providing these characteristics. The thickener found to impart the new and inventive characteristics of the present invention has been selected from the group of non-ionic, hydrophobic modified polymers, preferably a polyester from copolymer of PEG-180, Dodoxinol-5, PEG-25 tristyriphenol and monomers of tetrametoxymethylglycoury of the chemical class of synthetic polymers, the function of which is to act as an aqueous agent for intensifying viscosity. Preferably, the thickener is a polyether that has the following structural formula:

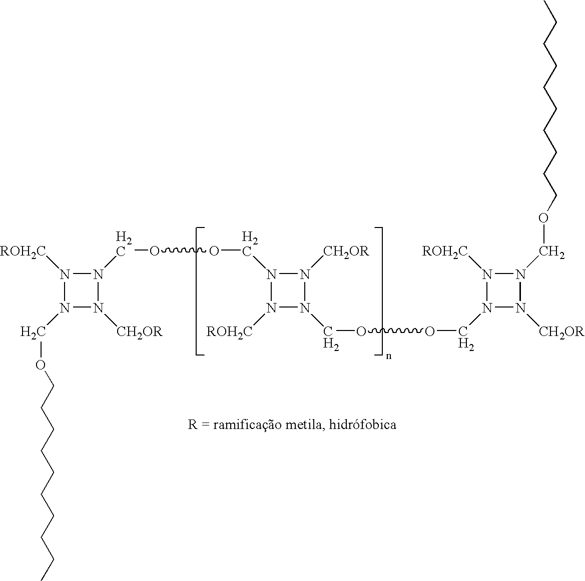

R=hydrophobic methyl branch

wherein n is greater than 1, preferably n could range in a way such that its final molecular is between 30,000 to 40,000.

Particularly, the thickener employed in the present invention is Polyether-1, commercially available as PURE THIX 1442™ and/or PURE-THIX HH™ from Sud-Chemie Rheologicals.

In addition to the effective characteristics inherent in the viscosity, compositions of this type have to provide a mixture that imparts softness and render the easiness of hair. For such a mixture of fatty alcohol+cationic surfactant is added. Said mixture may be selected from mixtures of fatty alcohol+cationic surfactant known in the state of the art. Particularly, the fatty alcohol+cationic surfactant mixture is a mixture of Cethyl Alcohol+Cethyltrimethyl Ammonium Chloride. Preferably, the fatty alcohol+cationic surfactant mixture is the mixture commercially available as GENAMIN CTAC-CP™ from Clariant.

The compositions of the present invention further comprise a silicones+cationic polymer mixture. The cationic polymer, in particular its cationic parts, imparts to the formula of the cream of the present invention the capacity of entangling or disentangling the hair, since it acts as an entangling agent. The choice of the cationic polymer was due to the characteristics of its polymeric chain, which impart more softness to the hair and allow a suitable film to be formed. The silicones employed in the compositions of the present invention have the function of a conditioning agent, brightness enhance, softness and combability.

The cationic polymer employed in the formulation of the present invention may be selected from the group consisting of polymers containing primary, secondary, tertiary and/or quaternary amine groups, forming part of the polymer chain or directly linked to the last one, having a molecular weight ranging from 500 to about 5,000,000.

There may be mentioned, without implied limitation:

quaternary polyammonium polymers such as those described in Application EP-A-122,324 or in French Patents 2,333,012, 2,270,846, 2,270,851, 2,471,777, 2,316,271 and 2,331,323 or U.S. Pat. No. 4,157,388.

Especially preferred quaternary polyammonium compounds are those containing the units:

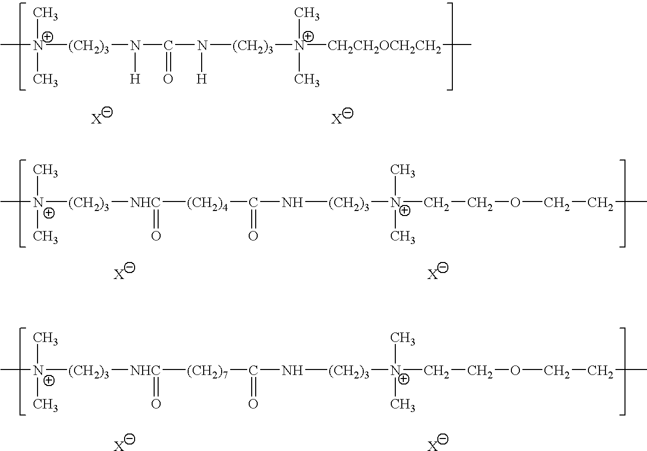

These quaternary polyammonium compounds are sold under the name MIRAPOL A15, MIRAPOL AD1, MIRAPOL AZ1 or MIRAPOL 175 by the company MIRANOL.

Cationic polysaccharides such as those described in U.S. Pat. Nos. 3,589,578 and 4,031,307, for example the product marketed under the name JAGUAR C 13S by MEYHALL may also be mentioned as cationic polymers.

Particularly, the cationic polymer is selected from Poliquaternium 10, of natural source obtained from hydroxyethylcellulose, a natural raw material that allows its quaternization through chemical processes. Poliquaternium 10 used in the present invention presents the following structural formula:

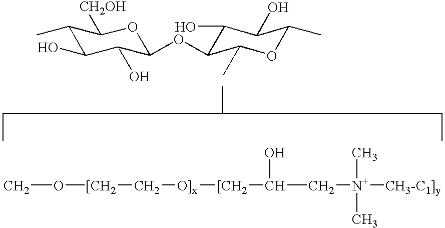

The family of cationic polymers UCARE™ POLYMERS that may be used in the formulations of the present invention is as follows:

| | | | Nitrogen | |
|---|---|---|---|---|
| Product | MW | Cationicity | (%) | Substantiality |
| Polymer JR | Low | ++ +++++ | 1.5-2,. | High |
| 125 | | | | |
| Polymer LK | Medium | ++ | 0.4-0.6 | Low |
| Polymer | Medium | ++ ++ | 0.8-1.1 | Medium |
| LR400 | | | | |
| Polymer JR | Medium | ++ +++++ | 1.5-2.2 | High |
| 400 | | | | |
| Polymer LR | High | ++ ++ | 0.8-1.1 | Medium |
| 30M | | | | |
| Polymer JR | High | ++ +++++ | 1.5-2.2 | High |
| 30M | | | | |

Preferably, the cationic polymer used in the present invention is the commercially available UCARE™ POLYMER LK from Amerchol.

The silicones employed in the formulation of the present invention may be soluble or insoluble silicones. Particularly, the silicones used are selected from the group of polyorganosiloxanes, which are insoluble in the composition and may take on the form of oils, waxes, resins and gums.

The silicones employed may be volatile or nonvolatile.

When volatile, the organopolysiloxylanes are selected from:

(a) cyclic silicones containing from 3 to 7 silicon atoms, and preferably 4 to 5. They are, for example, the octamethylcyclotetrasiloxane sold under the name “VOLATILE SILICONE 7207” by UNION CARBIDE or “SILBIONE 70045 V 2” by RHONE POULENC, or the decamethylcyclopentasiloxane sold under the name “VOLATILE SILICONE 7158” by UNION CARBIDE or “SILBIONE 70045 V 5” by RHONE POULENC, as well as mixtures thereof.

Cyclic copolymers of the dimethylsiloxane/methylalkylsiloxane type, such as “SILICONE VOLATILE FZ 3109” sold by the company UNION CARBIDE, of chemical structure:

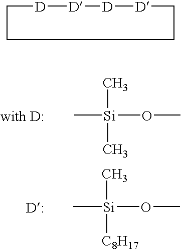

are also mentioned.

Mixtures of cyclic silicones with organic compounds derived from silicon, such as a mixture of octamethylcyclotetrasiloxane and tetratrimethylsilylpentaerythritol (50:50) and a mixture of octamethylcyclotetrasiloxane and 1,1′-oxy-2,2,2′,2′,3,3′-hexa(trimethylsilyloxy)bisneopentane may also be mentioned;

(b) volatile linear silicones having 2 to 9 silicon atoms and possessing a viscosity not exceeding 5×10⁻⁶ m²/s at 25° C. They are, for example, the decamethyltetrasiloxane sold under the name “SH 200” by the company TORAY SILICONE. Silicones belonging to this class are also described in the paper published in Cosmetics and Toiletries, Vol. 91, January 76, p. 27-32-TODD & BYERS “Volatile silicone fluids for cosmetics”.

Non-volatile silicones are preferably used, and more especially polyalkylsiloxanes, polyarylsiloxanes, polyalkylarylsiloxanes, silicone gums and resins and polyorganosiloxanes modified with organic functional groups, as well as mixtures thereof.

Among these polyalkylsiloxanes, there may be mentioned, without implied limitation, the following commercial products:

SILBIONE oils of the 47 and 70 047 series marketed by RHONE POULENC, such as, for example, the oil 47 V 500 000;

Oils of the 200 series of the company DOW CORNING;

The VISCASIL oils of GENERAL ELECTRIC and some oils of the SF series (SF 96, SF 18) of GENERAL ELECTRIC.

In this class of polyalkylsiloxanes, the products sold under the names “ABIL WAX 9800 and 9801” by the company GOLDSCHMIDT, which are poly (C.sub.1-C.sub.20 alkyl)siloxanes, may also be mentioned.

Among these polyalkylarylsiloxanes, there may be mentioned, by way of example, the products marketed under the following names:

SILBIONE oils of the 70 641 series of RHONE POULENC;

Oils of the RHODORSIL 70 633 and 763 series of RHONE POULENC;

The oil DC 556 COSMETIC GRADE FLUID of DOW CORNING;

Silicones of the PK series of BAYER, such as the product PK 20;

Silicones of the PN and PH series of BAYER, such as the products PN 1000 and PH 1000;

Some oils of the SF series of GENERAL ELECTRIC, such as SF 1023, SF 1154, SF 1250, SF 1265.

The silicone gums which are usable according to the invention are, in particular, polydiorganosiloxanes having high molecular masses of between 200,000 and 1,000,000, used alone or as a mixture in a solvent. This solvent may be selected from volatile silicones, polydimethylsiloxane (PDMS) oils, polyphenylmethylsiloxane (PPMS) oils, isoparaffins, methylene chloride, pentane, dodecane, tridecane, tetradecane or mixtures thereof.

There may be mentioned, more especially, the following products:

Polydimethylsiloxane/methylvinylsiloxane gums,

Polydimethylsiloxane/diphenylsiloxane,

Polydimethylsiloxane/phenylmethylsiloxane,

Polydimethylsiloxane/diphenylsiloxane/methylvinylsiloxane.

The organopolysiloxane resins which are usable according to the invention are crosslinked siloxane systems containing the units:

R₂SiO_{2/2}, RsiO_{3/2} and SiO_{4/2} in which units R represents a hydrocarbon group possessing 1 to 6 carbon atoms or a phenyl group. Among these resins, the product sold under the name “DOW CORNING 593” or those sold under the names “SILICONE FLUID SS 4230 and SS 4267” by the company GENERAL ELECTRIC, and which are siloxanes of dimethyl/trimethylsiloxane structure, may be mentioned.

Substituted or unsubstituted amino groups, such as the products sold under the name GP4 Silicone Fluid and GP 7100 by the company GENESEE or the products sold under the names Q2 8220 and DC 929 by the company DOW CORNING. The substituted amino groups are, in particular, C₁-C₄ aminoalkyl groups.

Particularly, the silicones may be selected from Dimeticonol, Ciclometicona, Amodimeticona and/or mixtures thereof. Preferably, the silicones employed are those available on the market as DC-949™ and DC 2-1288™ from Dow Corning.

The formulations of the present invention may also comprise additional formulation aiding components such as: esters, ethers, polymers, carriers, natural additives, conditioning agents, preservatives, dyes, pH adjusters, emollients, preserving agents, fragrances, moisteners, sun-screens, vitamins, proteins, hydrants, any other additive commonly used in cosmetic compositions and/or mixtures thereof.

In a variant embodiment of the present of the present invention, non-rinse compositions for combing hair comprising said thickener, as well as said cationic polymer, may be formulated

The weight concentrations of the components (i) to (iv) in said formulations are the following:

(i) thickener: from 0.001 to 20.0%;

(ii) fatty alcohol+surfactant mixture: from 0.000001 to 1.0%;

(iii) cationic polymer: from 0.000001 to 1.0%;

(iv) silicone: from 0.000001 to 10.0%.

Particularly, the weight concentrations of the components (i) to (iv) in said formulations are the following:

(i) thickener: from 0.5 to 10.0%;

(ii) fatty alcohol+surfactant mixture: from 0.1 to 1.0%;

(iii) cationic polymer: from 0.001 to 0.1%;

(iv) silicone: from 0.1 to 2.0%.

Depending upon the cosmetic purposes, the compositions of the present invention may be in the most varied forms such as, for example, creams, emulsions, and/or pastes.

The present invention further discloses the use of effective amounts of said components (i) to (iv) in the preparation of non-rinse compositions for combing hair.

The following examples illustrate the invention, without, however, limiting its scope in any way.

### FORMULATION EXAMPLE 1

| Component: | w/w (%) |
|---|---|
| Water | q.s.p. 100 |
| Cationic Polymer | 0.1 |
| Thickener | 2.0 |

### FORMULATION EXAMPLE 2

| Component: | w/w (%): |
|---|---|
| Water | q.s.p. 100 |
| Thickener | 2.0 |
| Cationic surfactant | 0.16 |
| Fatty Acid | 0.64 |

### FORMULATION EXAMPLE 3

| Component: | w/w (%): |
|---|---|
| Water | q.s.p. 100 |
| Cationic Polymer | 0.0625 |
| Thickener | 1.9 |
| Silicone | 1.59 |
| Cationic surfactant | 0.08 |
| Fatty Acid | 0.32 |

### FORMULATION EXAMPLE 4

| Component: | w/w (%): |
|---|---|
| Water | q.s.p. 100 |
| Cationic Polymer | 0.0625 |
| Thickener | 2.0 |
| Silicone | 1.59 |
| Cationic surfactant | 0.16 |
| Fatty Acid | 0.64 |

### FORMULATION EXAMPLE 5

| Component: | w/w (%): |
|---|---|
| Water | q.s.p. 100 |
| Cationic Polymer | 0.03 |
| Thickener | 1.5 |
| Silicone | 1.55 |
| Cationic surfactant | 0.16 |
| Fatty Acid | 0.64 |

### FORMULATION EXAMPLE 6

| Component: | w/w (%): |
|---|---|
| Water | q.s.p. 100 |
| Cationic Polymer | 0.0625 |
| Thickener | 1.7 |
| Silicone | 1.73 |
| Cationic surfactant | 0.16 |
| Fatty Acid | 0.64 |

### FORMULATION EXAMPLE 7

| Component: | w/w (%): |
|---|---|
| Water | q.s.p. 100 |
| Cationic Polymer | 0.0625 |
| Thickener | 1.8 |
| Silicone | 1.55 |
| Cationic surfactant | 0.16 |
| Fatty Acid | 0.64 |

### FORMULATION EXAMPLE 8

| Component: | w/w (%): |
|---|---|
| Water | q.s.p. 100 |
| Cationic Polymer | 0.08 |
| Thickener | 1.8 |
| Silicone | 1.55 |
| Cationic surfactant | 0.16 |
| Fatty Acid | 0.64 |

1. A non-rinse composition for combing hair, characterised by comprising:
(i) a thickener; and
(ii) a fatty alcohol+cationic surfactant mixture at low concentration.

2. The composition according to claim 1, characterised by further comprising:
(iii) a cationic polymer.

3. The composition according to claim 2, characterised by further comprising:
(iv) a silicone.

4. A non-rinse composition for combing hair, characterised by comprising:
(i) a thickener; and
(ii) a cationic polymer.

5. The composition according to claim 1 or 4, characterised in that said thickener is selected from the group of non-ionic, hydrophobic modified polymers.

6. The composition according to claim 5, characterised in that said thickener is a polyether of a copolymer of PEG-180, of Doxinol-5, of PEG-25 tristirylphenol and of tetrametoxymethylglycouryl monomers, of the chemical class of synthetic polymers, the function of which is an aqueous viscosity-intensifying agent.

7. The composition according to claim 6, characterised in that said thickener is Polyether-1, which represents the following structural formula: 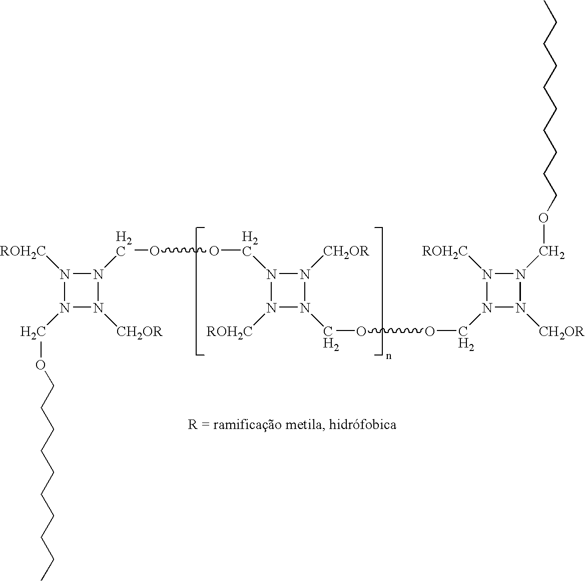
R=hydrophobic methyl branch
wherein n is greater than 1, preferably n could range in a way such that its final molecular is between 30,000 to 40,000.

8. The composition according to claim 2 or 4, characterised in that the cationic polymer is selected from the group consisting of Polymer JR 125, Polymer JR 400, Polymer JR 30M, Polymer LR 400, Polymer LR 30M and polymer LK and/or mixtures thereof.

9. The composition according to claim 8, characterised in that said cationic polymer is Poliquaternium 10, which presents the following structural formula:

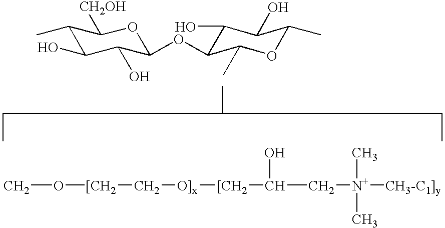

10. A composition according to claim 3, characterised in that the silicones are soluble or insoluble, and are selected from Dimethiconol, Cyclometicone, Amodimethicone and/or mixtures thereof.

11. The composition according to claim 1, characterised in that the fatty alcohol+surfactant mixture is a mixture of Cethyl Alcohol+Cethyltrimethyl Ammonium Chloride.

12. The composition according to any one of claims 1-11, characterised by comprising (weight %):
(i) thickener: from 0.001 to 20.0%
(ii) fatty alcohol+surfactant mixture: from 0.000001 to 1.0%;
(iii) cationic polymer: from 0.000001 to 1.0%;
(iv) silicone: from 0.000001 to 10.0%.

13. The composition according to claim 12, characterised by comprising (weight percent):
(i) thickener: from 0.5 to 10.0%;
(ii) fatty alcohol+surfactant mixture: from 0.1 to 1.0%;
(iii) cationic polymer: from 0.001 to 0.1%.
(iv) silicone: from 0.1 to 2.0%.

14. The composition according to any one of claims 1 to 13, characterised by further comprising additional formulation aiding components, selected from: esters, ethers, polymers, carriers, natural additives, conditioning agents, preservatives, dyes, pH adjusters, emollients, preserving agents, fragrances, moisteners, sun-screens, vitamins, proteins, hydrants, any other additive commonly used in cosmetic compositions and/or mixtures thereof.

15. The composition according to any one of claims 1 to 14, characterised in that it is in the form of creams, emulsions, and/or pastes.
